(19) United States
(12) Patent Application Publication
(10) Pub. No.: US 2011/0200994 A1
(43) Pub. Date: Aug. 18, 2011

(54) Method for Diagnosing a Genetic Predisposition for Vascular Disease

(76) Inventors: Irmgard Tegeder, Frankfurt (DE); Jörn Lötsch, Frankfurt am Main (DE); Keith M. Channon, Oxford (GB); Charalambos Antoniades, Oxford (GB)

(21) Appl. No.: 12/745,838

(22) PCT Filed: Dec. 2, 2008
(86) PCT No.: PCT/DE2008/001990

§ 371 Date: Feb. 24, 2011

(30) Foreign Application Priority Data
Dec. 3, 2007 (DE) 10 2007 058 340.2

(51) Int. Cl. C12Q 1/68; C07H 21/00; C07H 21/04

(52) U.S. Cl. 435/6.11; 536/24.33; 536/24.31

(57) ABSTRACT

The invention relates to a method for diagnosing a genetic predisposition for a vascular disease, in particular for a coronary artery disease (CAD). According to the invention, the method has the following steps: Providing a sample containing a nucleic acid derived from the gene GCH1; and determining the presence or absence of a nucleotide polymorphism (SNP) of the gene GCH1 in the nucleic acid, wherein the SNP is selected from the group consisting of rs8007267 G>A, rs3783641 A>T, and rs10483639 C>G, wherein the presence of at least one of said SNPs indicates a genetic predisposition for a vascular disease.

The invention relates to a method for diagnosing a genetic predisposition for a vascular disease, in particular for a coronary artery disease (CAD), and the use of said method for diagnosing a genetic predisposition for a vascular disease in an individual. Furthermore, the invention relates to a kit for diagnosing a genetic predisposition for a vascular disease, and the use of a respective kit.

The maintenance of the endothelial function represents a critical aspect of vascular homeostasis. The loss of the normal endothelial production of nitric oxide (NO) represents an early and characteristic feature of vascular diseases, and most likely plays a role in the pathogenesis of such diseases. The endothelial nitric oxide synthase (eNOS) is regulated by the co-factor tetrahydrobiopterin (BH4) (1 to 3). A reduced availability of BH4 due to a disease seems to represent an important aspect of the observed impaired eNOS-activity and the increased vascular peroxide production in animal models (1, 3). In humans with vascular diseases, the acute gavage of BH4 can improve the endothelial function (4, 5), and treatments with folates or vitamin C, which improve the endothelial function, possibly mediate at least parts of their effects by increasing the BH4-availability (6 to 8). Despite this, it still remains unclear as to what extent changes of the endogenous BH4-availability can play a direct role in the modulation of endothelial function in the human in vivo.

GTP-cyclohydrolase I (GTPCH) is the rate limiting enzyme in the biosynthesis of BH4 (9, 19), and due to the transcriptional regulation of the GCH1 expression as well as posttranslational modification, represents one of the most important determinants of the BH4 concentrations. In some experimental models, reduced BH4-concentrations were associated with reduced GTPCH-activity (11). Nevertheless, it is also possible that BH4 is sensitive for the oxidation by free radicals, such as, for example, peroxynitrite (ONOO⁻), whereby dihydrobiopterin (BH2) and biopterin (B) are generated without that a significant change in the concentration of the overall-biopterins can be found (6, 8). The relative importance of the synthesis of BH4, compared with the oxidation of BH4, in the context of arteriosclerosis is complex, since a local and symmetric inflammation leads to an up-regulation of the GCH1 expression (12), but also increases the ONOO⁻-production, which possibly leads to an increased BH4-oxidation (13).

### DESCRIPTION OF THE INVENTION

In order to be independent from the difficult interpretation of concentrations of intracellular metabolites, whose regulation in the cell is still largely unknown, a diagnosis based on genetic parameter would be desirable in the diagnosis of vascular diseases. Accordingly, it was the object of the present invention to allow the predisposition for a vascular disease in a simple and readily interpretable manner.

The object underlying the invention is solved by a method for diagnosing a genetic predisposition for a vascular disease, in particular for a coronary artery disease (CAD). According to the invention, the method comprises the following steps:
First, a sample is provided containing a nucleic acid which derived from the genomic locus of the gene GCH1. The term “derived nucleic acid” shall include the genomic DNA, as well as an unprocessed and therefore non-spliced RNA (also designated as hnRNA), and/or a cDNA derived therefrom.
In a second step of the method according to the invention, the presence or absence of a nucleotide polymorphism (SNP) of the gene GCH1 in the nucleic acid is determined. Thereby, the nucleotide polymorphism, which describes a specific GCH1 haplotype, is selected from the group consisting of rs8007267G>A, rs3783641A>T, and rs10483639C>G. The presence of at least one of said SNPs indicates a genetic predisposition for a vascular disease. In a preferred embodiment of the method according to the invention, the presence or absence of at least two, particularly preferred of all three nucleotide polymorphisms as indicated is determined.

The nomenclature of the SNPs as indicated is based on the dbSNP-accession number, which known to the person of skill.

Besides the determination of the presence or absence of at least one of the above-mentioned nucleotide polymorphisms of the gene GCH1 in the nucleic acid, also a determination of the presence or absence of an SNP, which is inherited as a block together with the haploblock comprising the above-mentioned nucleotide polymorphisms, can be used.

The nucleotide polymorphism rs80072670>A can be found in the 5′-non-translated region of the gene GCH1, whereas the nucleotide polymorphism rs3783641A>T is found in the intron 1, and the nucleotide polymorphism rs10483639C>G in the 3′-non-translated region of the gene GCH1. Due to this fact, the method according to the invention can only be performed with those nucleic acids, which map the mentioned nucleotide polymorphisms in the non-translated region of the gene.

Using the method according to the invention, individuals can be identified, which have a genetic predisposition for a vascular disease, in particular for a coronary artery disease. Thereby, in the individuals as affected, the nucleotide polymorphisms as mentioned affect the following pathobiochemical parameters: One the one hand, these individuals show a reduced GCH1 expression in the walls of the vessels. Furthermore, a reduced concentration of biopterins, in particular of BH4, can be found in the blood plasma and in the blood vessel walls. Finally, the individuals as affected show a modified regulation of the endothelial nitric oxide synthase (eNOS) in the cells of the blood vessels auf. The latter leads to a reduced concentration of nitric oxide (NO) in the blood plasma and in den walls of the vessels, to an increased vascular superoxide production, as well as a reduction of the acetylcholine-mediated vasorelaxation, wherein the latter points to an endothelial dysfunction. The terms “reduced”, “lowered”, “decreased” and “increased” as used each refer to a statistically significant deviation compared with a healthy individual, which does not have the nucleotide polymorphisms as mentioned.

In a preferred embodiment of the method, the sample originates from a mammal, in particular from a human.

In a preferred embodiment of the method according to the invention the detection of the presence or absence of an SNP of the gene GCH1 in the nucleic acid is carried out by sequencing, primer extension, hybridization, restriction-fragment-analysis, oligonucleotide ligation and/or an allele-specific PCR.

In most applications, it will be advantageous to select a detection method, by means of which a high-throughput genotyping can be performed. For this, generally four different mechanisms are available, namely the allele-specific primer extension, the allele-specific hybridization, the allele-specific oligonucleotide ligation, and the allele-specific cleavage of a “flap probe” (42). The sequencing or the primer extension technology, respectively, is based on the determination of the sequence at a particular base position, wherein the extension of a primer at this particular site only occurs, if a particular base is present in the template (Landegren et al., Genome Res. 8, 769 to 776 (1998)). Allele-specific hybridization protocols are based on the recognition of one or several SNPs. For this, different techniques were developed, which are described, for example, in the following documents (Livak, Genet Anal. 14, 143 (1999); Tyagi et al., Nat. Biotechnol. 16, 49 (1998)).

Hybridization is used for genotyping also in connection with so-called microchips, which are described several publications (Hacea et al., Nature Genetics 14, 441 (1996); Shoemaker et al., Nature Genetics 14, 450 (1996); Chee et al., Sciences 274, 610 (1996); DeRisi et al., Nature Genetics 14, 457 (1996); Fun et al., Genome Res. 10, 853 (2000)).

Allele-specific oligonucleotide ligation assays have the advantage of a high specificity, in particular, since these methods can be used, for example, together with the fluorescence-resonance-energy-transfer (FRET)—technology (Chen et al., Genome Res. 8, 549 (1998)).

The presence of one of the polymorphisms as mentioned can generally be detected using any type of body cell. It is particularly advantageous, if the sample as provided is obtained from a body fluid, since it is in general readily obtainable using only minimally invasive methods. Suitable body fluids include blood, ejaculate, saliva, bronchial lavage, pleural effusion, peritoneal fluid, amniotic fluid or oral mucosal swab. In an alternative embodiment of the method according to the invention, the sample as provided id obtained from a tissue, such as, for example, a blood vessel.

The problem underlying the invention is also solved by a kit for diagnosing a genetic predisposition for a vascular disease, in particular for a coronary artery disease. This kit can be used for carrying out the above-mentioned method. According to the invention, such a kit comprises at least one probe and/or at least one primer for a detection of at least one nucleotide polymorphism of the gene GCH1 auf, wherein the nucleotide polymorphism is selected from the group consisting of rs8007267G>A, rs3783641A>T, and rs10483639C>G.

In a preferred embodiment, the primer of the kit is an amplification primer or a sequencing primer, so that the respective nucleotide polymorphism can be determined using amplification, for example in the context of a polymerase chain reaction, or by means of sequencing. The probe or the primer can be labeled with a suitable label, so that the kit, for example, allows to carry out a real time-PCR. In an alternative embodiment, the kit comprises a probe which is arranged on a solid basis, so that a biochip is present.

The preferred primers for PCR amplification of the GCH1 gene segments according to the invention and for sequencing are given in the following.

PCR primer for:

dbSNP rs8007267 G > A:
forward:
(SEQ ID No. 1)
5′-TGGGGTGAGGGTTGAGTT-3′,
reverse:
(SEQ ID No. 2)
5′-biotin-AATGTTAACACAATAGGAGCG-3′;
dbSNP rs3783641 A > T:
forward:
(SEQ ID No. 3)
5′-GCTATTTGCTTTGTCCACCTCTA-3′,
reverse:
(SEQ ID No. 4)
5-biotin-AACCTGGAACTGAGAATTGTTCAC-3′;
dbSNP rs10483639 C > G:
forward:
(SEQ ID No. 5)
5′-ATCCTTTCAATCTGGAACTGACTG-3′,
reverse:
(SEQ ID No. 6)
5′-biotin-GCATTCTAAAATCAGGGAAAATCA-3′.

Sequencing primer for:

dbSNP rs8007267 G > A:

| | |
|---|---|
| 5′-CTTGAATGACTGAAGTTTGG-3′; | (SEQ ID No. 7) |
| dbSNP rs3783641 A > T: | |
| 5′-CCCACCTGACTCATTT-3′; | (SEQ ID No. 8) |
| dbSNP rs10483639 C > G: | |
| 5′-GGTGTGTGTATGTACAACTT-3′. | (SEQ ID No. 9) |

The problem underlying the invention is also solved by the use of one of the methods as described above for diagnosing a genetic predisposition for a vascular disease, in particular for a coronary artery disease (CAD). Besides the use of the method as mentioned for a diagnosis, said can also be used, for example, in research.

The invention will be further explained in the following on the basis of examples, without being limited to these examples. The results of the experiments as described in the examples are depicted in the figures.

### FIGURES

The Figures show:

FIG. 1: The presence of the rare haplotype (XO or XX) was associated with significantly lower concentrations of plasma tetrahydrobiopterin (BH4, a) and total biopterin (tBio, b). A similar effect could also be observed in saphenous veins (SV, c and d) and internal arteries of the mamma (IMA, e, and f).

FIG. 2: The presence of the XX-genotype (homozygous for the GCH1-polymorphism), when compared with the OO-genotype (homozygous wildtype CGH1) was associated with significantly lower mRNA-concentrations of GTPCH. Results from experiments are shown which were obtained with 23 saphenous veins (SV) and 17 internal arteries of the mamma (IMA). *P<0.05 vs OO.

FIG. 3: The presence of the X-haplotype was associated with a higher total superoxide (O2⁻) production (a) and larger L-NAME-inhibitable delta(O2⁻) (b) in human saphenous veins. The X-haplotype was also associated with a significantly lower plasma BH4:tbio-quotient (c) and higher plasma concentrations of oxidized LDL (ox-LDL, d). Furthermore, the X-haplotype was associated with a lower maximal vasorelaxation in response to acetylcholine (ACh, e) in saphenous veins. In contrast, the X-haplotype had no impact on the vasomotoric response to sodium nitroprusside (SNP, f).

TABLE 1
Demographic characteristics of the patients

| | |
|---|---|
| Number of patients | 347 |
| Male/female | 292/55 |
| Age (Years) | 65.42 ± 0.49 |
| Saphenous veins (n) | 289 |
| Internal arteries of the mamma (n) | 166 |
| Risk factors | |
| High blood pressure (%) | 72.7 |
| Hypercholesterolemia (%) | 71.6 |
| Smoker (current/former) (%) | 8.6/69.8 |
| Diabetes Mellitus (%) | 27.5 |
| Familiar history of coronary artery diseases (%) | 63.3 |
| Body mass index (Kg/m²) | 28.04 ± 0.34 |
| Triglycerides (mmol/l) | 1.65 ± 0.09 |
| Cholesterol (mmol/l) | 4.07 ± 0.08 |
| High density lipoproteins, HDL, (mmol/l) | 1.10 ± 0.02 |
| C-reactive protein (mg/l) | 2.48 ± 0.07 |
| Plasma biopterin concentrations | |
| Plasma-tetrahydrobiopterin (nmol/l)^{#} | 20.46 [10.13-41.86] |
| Plasma-dihydrobiopterin (mmol/l)^{#} | 15.19 [11.77-19.41] |
| Plasma-biopterin (mmol/l)^{#} | 4.09 [2.83-6.98] |
| Plasma-Gesamt-biopterin (mmol/l)^{#} | 46.11 [30.04-71.33] |
| BH4/tbio-quotient | 0.49 ± 0.018 |
| Medication (%) | |
| Statine | 90.0 |
| ACEi/ARB | 66.0 |
| Potassium channel blocker | 40.8 |
| B-blocker | 74.6 |
| Aspirin | 87.1 |

ACEI: inhibitors of the angiotensin-converting enzyme;
ARB: angiotensin receptor blocker;
values expressed as mean value ± standard deviation of the mean value;
^{#}values expressed as median (25.-75. percentile).

### EXAMPLES

### Materials and Methods

### Test Persons

347 patients with CAD, which were subjected to coronary bypass-surgery (CABG), were examined at the John Radcliffe Hospital, Oxford, UK. Any inflammatory, infectious, liver or kidney disease, malignant disease, any acute coronary incidence within the last 2 months or any clinically obvious heart failure was regarded as exclusion criteria. Furthermore, patients were excluded, which took non-steroidal anti-inflammatory medicaments, or dietary supplements with folic acid or anti-oxidative vitamins. The demographic characteristics of the patients are shown in Table 1. The study was approved by the responsible research ethics committee, and every included patient gave written informed consent.

### Plasma and Tissue Samples

Blood samples were drawn after fasting over night directly before the surgery. The samples were centrifuged at 2.500 rpm for 10 minutes, and serum or plasma were stored at −80° C. until examination.

Samples from saphenous veins (SV) and the internal artery of the mamma (arteria mammaria interna; IMA) were taken as described at the time of CABG surgery (6, 20, 21). For measuring the biopterin content, paired vessel segments were frozen in liquid nitrogen and stored at −80° C., or were shipped to the laboratory for functional studies within 30 minutes in ice-cold Krebs-Henseleit buffer (6, 20, 21).

### DNA Extraction and Genotyping

Genomic DNA was extracted from 2 ml of whole blood using standard methods (QIAamp DNA blood Midi Kit, Qiagen, Germantown, Md.). The GCH1 haplotype was diagnosed using the validated Pyrosequencing™ assay through typing of 3 GCH1 nucleotide polymorphisms (“single nucleotide polymorphisms”) (dbSNP rs8007267G>A, dbSNP rs3783641A>T and dbSNP rs10483639C>G) as described (18, 19).

RNA Isolation and Realtime Quantitative Polymerase Chain Reaction (RT qPCR)

Vascular rings (IMA and SV) frozen in liquid nitrogen were first lysed using Trizol®-reagent, and subsequently RNA was purified from the aqueous phase using the RNeasy Micro Kit (Qiagen). RNA was converted into cDNA (Superscript IT reverse transcriptases, Invitrogen) and then subjected to a quantitative PCR using the TaqMan system (Applied Biosystems Assay ID GCH=Hs00609198_m1, Assay ID GAPDH=Hs02758991_g1), and analyzed on an iCyclerIQ (Biorad). The relative expression was calculated using the 2^{-ΔC(T)} method, which is described in (22).

### Determination of the Biopterin Concentrations in Plasma and in the Vessel Wall

Concentrations of BH4, BH2 and biopterin in plasma or vessel wall lysates were each determined independently of each other from the same sample, namely by HPLC followed by serial electrochemical and fluorescence detection, as described in (23). Total-biopterins (tBio) were quantified by pooling of BH4, BH2 and B. Biopterin concentrations were expressed as pmol/g tissue for vessels and nmol/l for plasma.

### Determination of the Vascular Superoxide Production

Vascular superoxide production was measured in paired segments of SV as described (6, 24), using lucigenin-enhanced chemiluminescence. Vessels were opened longitudinally, in order to expose the endothelial surface, and equilibrated for 20 minutes in oxigenised (95% O₂/5% CO₂) Krebs-HEPES buffer (pH=7.4) at 37° C. Lucigenin-enhanced chemiluminescence was measured using low-concentrated lucigenin (5 μmol/l) (21). As a measure for “eNOS coupling”, the NOS-mediated superoxide production was measured, which was estimated as the difference in the superoxide production after 20 minutes of incubation with the NOS-inhibitor N^{G}-Nitro-L-arginine methyl ester (L-NAME; 100 μmol/l).

### Vasomotoric Studies

Using isometric tension studies, the endothelium-dependent and endothelium-independent dilatation was in determined in SV which were obtained at the time of CABG (6, 20, 21). From each vessel four rings were each pre-contracted with phenylephrin (3×10⁻⁶ mol/l), and then the endothelium-dependent relaxation was quantified using acetylcholine (ACh, 10⁻⁹ mol/l to 10⁻⁵ mol/l). Finally, as already described (6, 20, 21), the relaxation as a reaction to the endothelium-independent NO-donor sodium nitroprusside (SNP, 10⁻¹⁰ mol/l to 10⁻⁶ mol/l) in the presence of L-NAME (100 μmol/l) was determined.

### Determination of the Concentrations of Oxidized LDL and C-Reactive Protein

Serum concentrations of the oxidized LDL (ox-LDL) were measured using enzyme-bound immunosorbent assay (ELISA) using commercially available kits (Mercodia, Sweden). Serum concentrations of the C-reactive protein (CRP) were measured using immuno-nephelometry using a highly sensitive method (Dade Behring Marburg GmbH, Marburg, Germany).

### Statistical Analysis

All variables were tested for normal distribution using the Kolmogorov-Smirnov test. Normally distributed variables are depicted as mean value±standard deviation of the mean value, whereas non-normally distributed variables were logarithmically transformed for the analysis, and are given as median (25. to 75. percentile), together with the range. The comparison between the base line and the demographic characteristics between the three genotypes was performed using “one-way ANOVA” for multiple comparisons, whereas an unpaired t-test was used for comparing variables between two groups (for recessive models).

An univariate analysis was performed by means of calculating the Pearson's coefficient. A multivariate analysis was used for analyzing the effect of the genotype on the vascular biopterin or plasma-biopterin, respectively, vascular superoxide, L-NAME-induced changes of vascular superoxide or maximal relaxations on ACh as dependent variables. As independent variables, GCH1 genotype and the clinical characteristics (age, gender, diabetes mellitus, smoking, dyslipidemia, high blood pressure, body weight index and administrations of medicaments), which showed an association with the dependent variables in the univariate analysis at a level of 15%, were used. A reverse elimination method was used in all models, with P >0.1 as threshold for a removal of a variable from the model. All P-values were determined with a two-sided test, and P<0.05 was regarded as statistically significant. All statistical analyses were performed using SPSS 12.0.

### Results

The GCH1 haplotype was examined in 347 patients with coronary heart disease. The concordance of the three SNPs used for the definition of the haplotype was 100%. Allele-frequencies of the O and X haplotype were 84.3% and 15.7%, respectively, whereby the genotype-frequencies of OO were 70.6%, of XO 27.4%, and of XX 2.0%. This is in agreement with the Hardy-Weinberg-distribution and with studies as performed in other cohorts (18, 19).

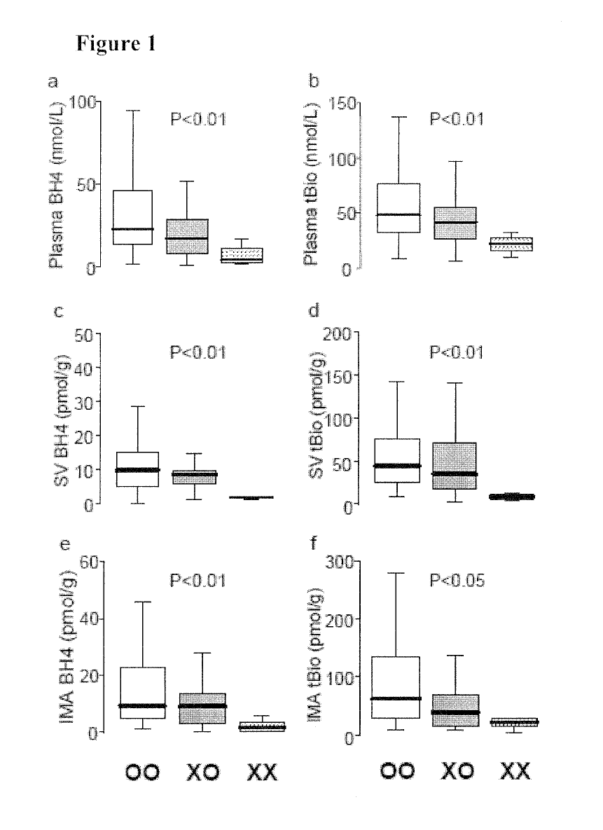

First, the effect of the GCH1 haplotype on the concentration of plasma-biopterin was examined. Patients with the haplotype X showed significantly lower plasma concentrations, both of BH4 as well as of total-biopterin, when compared with patients, which are homozygous for the common O haplotype. The median of the plasma-BH4-concentrations in patients with the XX genotype was reduced by about 80% compared with the OO patients. Both plasma-BH4-as well as total-biopterin-concentrations were reduced in a strongly allele-dependent fashion by the OO, XO, and XX genotype (FIG. 1).

Since it is likely that vascular biopterin-concentrations are more important for the eNOS-regulation in the endothelium, and these possibly are regulated different from plasma-biopterin-concentrations, in the following the effect of the GCH1 haplotype on vascular biopterin-concentrations both in SV as well as in IMA was examined. The presence of the X haplotype was associated with significantly lower concentrations of vascular BH4 and tbio, both in SV and IMA (FIG. 1). These results are evidence for a direct effect of the GCH1 haplotype on the biopterin-synthesis.

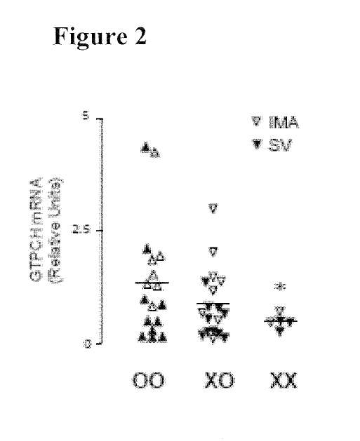

I order to examine, whether the effect of the GCH1 haplotype on the biopterin-concentrations was caused by changes in the GCH1 gene expression, using QRT-PCR the GCH1 mRNA was quantified in SV and IMA samples of patients with different GCH1 genotypes. It was observed that the XX genotype was associated with a significantly reduced vascular GCH1 expression, when compared with the OO genotype (FIG. 2). Furthermore, vascular GCH1 mRNA concentrations were correlated across all genotypes with BH4-concentrations both in plasma (r=0.394, P=0.010), and vascular (r=0.336, P=0.042).

It was furthermore examined, whether these genetically determined differences in the biopterin-concentrations could affect the NO-mediated endothelial function and vascular superoxide production. Since the homozygous X haplotype is rare with 2% in the population, a recessive model was used, wherein the patients with and without X haplotype were compared. Patients with the X haplotype (e.g. XO or XX genotype) showed significantly lower BH4- and total-biopterin-concentrations than OO patients, both in plasma (16.6 [5.2-28.3] and 41.2 [23.4-54.9] vs 21.57 [12.1-45.24], and 48.3 [32.6-76.4] nmol/l, P<0.05 for both) and in SV (0.71 [0.22-0.93], and 3.27 [0.22-0.93] vs 1.10 [0.57-1.78] and 4.3 [2.4-7.6] nmol/l, P<0.05 for both).

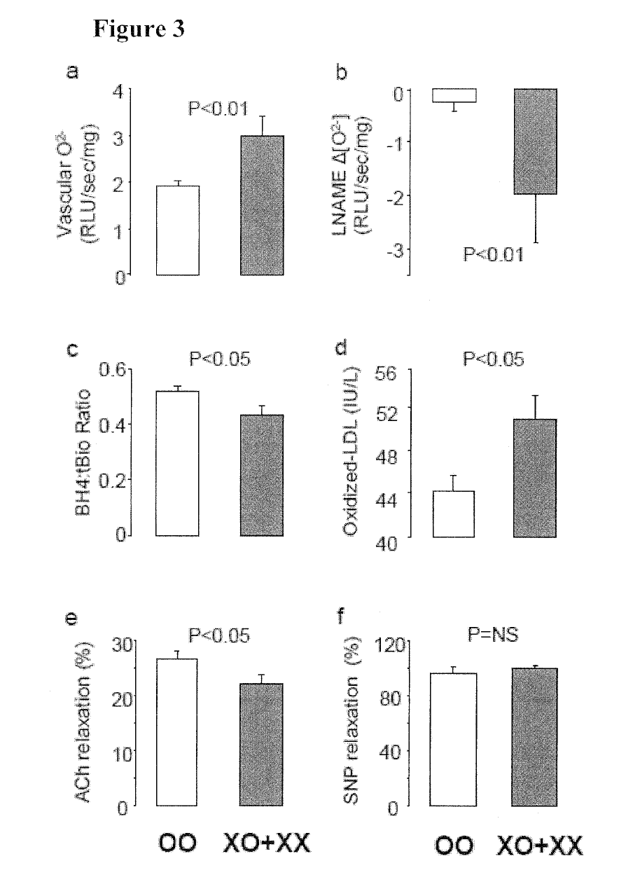

In carriers of the X haplotype the vascular superoxide production was significantly increased (FIG. 3). In order to examine the specific contribution of the “eNOS coupling”, the differences in the vascular superoxide production after NOS-inhibition through L-NAME were quantified. The incubation of vessels with L-NAME reduced the superoxide production to a significantly larger extent in carriers of the X haplotype, compared with the O-homozygotes (FIG. 3). The assumption of an increased oxidative stress in carriers of the X haplotype was also supported by the measurement of a lower BH4:total-biopterin-quotient and lower concentrations of oxidized LDL (FIG. 3).

In order to examine, how the GCH1 haplotype and the differences in the biopterin-concentrations as associated therewith modulate the NO-mediated endothelial function, the vasomotoric response of vessel segments to ACh was quantified in an organ-bath-system. Vasorelaxation-responses to ACh were significantly reduced in carriers of the X haplotype compared with O-homozygotes, whereas endothelium-independent vasomotoric responses to the immediate NO-donor, SNP, were identical between both genotypes (FIG. 3).

### Multivariate Analysis

BH4-concentrations and endothelial function can be modified by many factors. Thus, a multivariate analysis approach was chosen in order to analyze the ratio between GCH1 haplotype and biopterin-concentrations, whereby other clinical demographic factors were included. The X haplotype was identified as an independent predictor both for plasma and vascular BH4-concentrations (plasma: β(SE)-11.764 (4.722), P=0.014; SV: β(SE) −0.306 (0.147), P=0.039). Furthermore, the X haplotype was independently associated with the vascular superoxide production (SV: β(SE) 1.196 (0.573), P=0.04), and furthermore the maximal relaxation in reaction to ACh (β(SE) −5.304 (2.355), P=0.026).

### Discussion

Although BH4 is an essential cofactor for the maintenance of a functional eNOS-status in the vascular endothelium, only little is known about the pathophysiological control of endothelial BH4-concentrations in the human. In experimental animal models, atherosclerotic risk factors, such as diabetes mellitus (16), high blood pressure (25) or dyslipidemia (26) were associated with vascular BH4-deficiency, an effect, which is mainly mediated by an intracellular oxidation of BH4 into BH2 and B by means of reactive oxygen species (such as peroxynitrite) (8, 27). Although oxidative stress apparently represents an important regulatory mechanism for vascular BH4-bioavailability, the mechanisms that control the biosynthesis of BH4 in vascular diseases remain in dispute.

The biosynthesis of BH4 starts with. GTPCH, which catalyses the conversion of GTP into dihydroneopterine triphosphate, which is then converted into BH4 by the 6-pyruvoyl tetrahydrobiopterin synthase and sepiapterine reductase (28). In contrast to the latter enzymes, the activity of GTPCH in most tissues is rate-limiting (29) and is often regarded as the main regulator of BH4-synthesis. The GTPCH-encoding GCH1 gene is expressed in different types of cells, such as macrophages (29), endothelial cells (30), and other cells. Experimental studies suggest that the GCH1 expression in endothelial cells or macrophages can be induced by different stimuli, such as insulin (31), stress by shear forces (32), and inflammation (33, 34). Using experimental mouse models based on changes in the vascular specific GCH1 expression (9, 15) it could be shown that GTPCH is a key regulator of the vascular BH4-concentration in vivo. Despite all of this, until today it was unclear in as much a modified GCH1 expression in human vessel walls has an effect on vascular BH4-concentrationen, or in as much the die modified GCH1 expression has an effect on the endothelial function in patients with vascular diseases.

### REFERENCES

1. Harrison D, Griendling K K, Landmesser U, Hornig B, Drexler H. Role of oxidative stress in atherosclerosis. Am J Cardiol 2003; 91:7 A-11A.
2. Channon K M. Tetrahydrobiopterin: regulator of endothelial nitric oxide synthase in vascular disease. Trends Cardiovasc Med 2004; 14:323-7.
3. Stroes E, Kastelein J, Cosentino F, et al. Tetrahydrobiopterin restores endothelial function in hypercholesterolemia. J Clin Invest 1997; 99:41-6.
4. Heitzer T, Brockhoff C, Mayer B, et al. Tetrahydrobiopterin improves endothelium-dependent vasodilation in chronic smokers: evidence for a dysfunctional nitric oxide synthase. Circ Res 2000; 86:E36-E41.
5. Antoniades C, Shirodaria C, Warrick N, et al. 5-methyl-tetrahydrofolate rapidly improves endothelial function and decreases superoxide production in human vessels: effects on vascular tetrahydrobiopterin availability and eNOS coupling. Circulation 2006; 114:1193-1201.
6. Shirodaria C, Antoniades C, Lee J, et al. Global improvement of vascular function and redox state with low-dose folic acid: implications for folate therapy in patients with coronary artery disease. Circulation 2007; 115:2262-70.
7. Kuzkaya N, Weissmann N, Harrison D G, Dikalov S. Interactions of peroxynitrite, tetrahydrobiopterin, ascorbic acid, and thiols: implications for uncoupling endothelial nitric-oxide synthase. J Biol Chem 2003; 278:22546-54.
8. Stroes E S, van Faassen E E, Yo M, et al. Folic acid reverts dysfunction of endothelial nitric oxide synthase. Circ Res 2000; 86:1129-34.
9. Verhaar M C, Wever R M, Kastelein J J, van Dam T, Koomans H A, Rabelink T J. 5-methyltetrahydrofolate, the active form of folic acid, restores endothelial function in familial hypercholesterolemia. Circulation 1998; 97:237-41.
10. Khoo J P, Nicoli T, Alp N J, Fullerton J, Flint J, Channon K M. Congenic mapping and genotyping of the tetrahydrobiopterin-deficient hph-1 mouse. Mol Genet Metab 2004; 82:251-4.
11. Meininger C J, Marinos R S, Hatakeyama K, et al. Impaired nitric oxide production in coronary endothelial cells of the spontaneously diabetic BB rat is due to tetrahydrobiopterin deficiency. Biochem J 2000; 349:353-356.
12. Landmesser U, Dikalov S, Price S R, et al. Oxidation of tetrahydrobiopterin leads to uncoupling of endothelial cell nitric oxide synthase in hypertension. J Clin Invest 2003; 111:1201-9.
13. Huang A, Zhang Y Y, Chen K, IIatakeyama K, Keaney J F, Jr. Cytokine-stimulated GTP cyclohydrolase I expression in endothelial cells requires coordinated activation of nuclear factor-kappaB and Stat1/Stat3. Circ Res 2005; 96:164-71.
14. Alp N J, McAteer M A, Khoo J, Choudhury R P, Channon K M. Increased endothelial tetrahydrobiopterin synthesis by targeted transgenic GTP-cyclohydrolase I overexpression reduces endothelial dysfunction and atherosclerosis in ApoE-knockout mice. Arterioscler Thromb Vase Biol 2004; 24:445-450.
15. Cosentino F, Barker J E, Brand M P, et al. Reactive oxygen species mediate endothelium-dependent relaxations in tetrahydrobiopterin-deficient mice. Arterioscler Thromb Vase Biol 2001; 21:496-502.
16. Khoo J P, Zhao L, Alp N J, et al. A pivotal role for tetrahydrobiopterin in pulmonary hypertension. Circulation 2005; 111:2126-2133.
17. Alp N J, Mussa S, Khoo J, et al. Tetrahydrobiopterin-dependent preservation of nitric oxide-mediated endothelial function in diabetes by targeted transgenic GTP-cyclohydrolase I overexpression. J Clin Invest 2003; 112:725-735.
18. Tegeder I, Costigan M, Griffin R S, et al. GTP cyclohydrolase and tetrahydrobiopterin regulate pain sensitivity and persistence. Nat Med 2006; 12:1269-77.
19. Lotsch J, Belfer I, Kirchhof A, et al. Reliable Screening for a Pain-Protective Haplo-type in the GTP Cyclohydrolase 1 Gene (GCH1) Through the Use of 3 or Fewer Single Nucleotide Polymorphisms. Clin Chem 2007; 53:1010-5.
20. Guzik T J, Mussa S, Gastaldi D, et al. Mechanisms of increased vascular superoxide production in human diabetes mellitus: Role of NAD(P)H oxidase and endothelial nitric oxide synthase. Circulation 2002; 105:1656-1662.
21. Tegeder I, Costigan M, Griffin R S, et al. GTP cyclohydrolase and tetrahydrobiopterin regulate pain sensitivity and persistence. Nat Med 2006; 12:1269-1277.
22. Shi L, Norling L A, Lau A S, Krejci S, Laney A J, Xu Y. Real time quantitative PCR as a method to evaluate simian virus 40 removal during pharmaceutical protein purification. Biologicals 1999; 27:253-62.
23. Heales S, Hyland K. Determination of quinonoid dihydrobiopterin by high-performance liquid chromatography and electrochemical detection. J Chromatogr 1989; 494:77-85.
24. Skatchkov M P, Sperling D, Hink U, et al. Validation of lucigenin as a chemiluminescent probe to monitor vascular superoxide as well as basal vascular nitric oxide production. Biochem Biophys Res Commun 1999; 254:319-24.
25. Guzik T J, West N E J, Black E, et al. Vascular superoxide production by NAD(P)H oxidase: association with endothelial dysfunction and clinical risk factors. Circ Res 2000; 86:e85-e90.
26. Channon K M, Guzik T J. Mechanisms of superoxide production in human blood vessels: relationship to endothelial dysfunction, clinical and genetic risk factors. J Physiol Pharmacol 2002; 53:515-24.
27. Lewontin R C. The Interaction of Selection and Linkage. Ii. Optimum Models. Genetics 1964; 50:757-82.
28. Gaut B S, Long A D. The lowdown on linkage disequilibrium. Plant Cell 2003; 15:1502-6.
29. Barrett J C, Fry B, Mailer J, Daly M J. Haploview: analysis and visualization of LD and haplotype maps. Bioinformatics 2005; 21:263-5.
30. Hardy G H. Mendelian proportions in a mixed population. Science 1908; 28:49-50.
31. Thum T, Fraccarollo D, Schultheiss M, et al. Endothelial nitric oxide synthase uncoupling impairs endothelial progenitor cell mobilization and function in diabetes. Diabetes 2007; 56:666-74.
32. Laursen J B, Somers M, Kurz S. et al. Endothelial regulation of vasomotion in apoE-deficient mice: implications for interactions between peroxynitrite and tetrahydrobiopterin. Circulation 2001; 103:1282-8.
33. Shinozaki K, Nishio Y, Okamura I, et al. Oral administration of tetrahydrobiopterin prevents endothelial dysfunction and vascular oxidative stress in the aortas of insulin-resistant rats. Circ Res 2000; 87:566-573.
34. Shinozaki K, Hirayama A, Nishio Y, et al. Coronary endothelial dysfunction in the insulin-resistant state is linked to abnormal pteridine metabolism and vascular oxidative stress. J Am Coll Cardiol 2001; 38:1821-8.
35. Werner-Felmayer G, Werner E R, Fuchs D, Hansen A, Reibnegger G, Wachter H. Tetrahydrobiopterin-dependent formation of nitrite and nitrate in murine fibroblasts. J Experim Med 1990; 172:1599-1607.
36. Thou B, Auerbach G, Blau N. Tetrahydrobiopterin biosynthesis, regeneration and functions. Biochem J 2000; 347 Pt 1:1-16.
37. Werner E R, Werner-Felmayer G, Fuchs D, et al. Tetrahydrobiopterin biosynthetic activities in human macrophages, fibroblasts, THP-1, and T 24 cells. GTP-cyclohydrolase I is stimulated by interferon-gamma, and 6-pyruvoyl tetrahydropterin synthase and sepiapterin reductase are constitutively present. J Biol Chem 1990; 265:3189-92.
38. Linscheid P, Schaffner A, Blau N, Schoedon G. Regulation of 6-pyruvoyltetrahydropterin synthase activity and messenger RNA abundance in human vascular endothelial cells. Circulation 1998; 98:1703-6.
39. Antoniades C, Shirodaria C, Crabtree M, et al. Altered Plasma vs. Vascular Biopterins in Human Atherosclerosis Reveal Relationships Between Endothelial Nitric Oxide Synthase Coupling, Endothelial Function and Inflammation. Circulation 2008;(In press).
40. Zhang L, Rao F, Zhang K, et al. Discovery of common human genetic variants of GIP cyclohydrolase 1 (GCH1) governing nitric oxide, autonomic activity, and cardiovascular risk. J Clin Invest 2007; 117:2658-71.
41. Vasquez-Vivar J, Kalyanaraman B, Martasek P, et al. Superoxide generation by endothelial nitric oxide synthase: the influence of cofactors. Proc Natl Acad Sci USA 1998; 95:9220-5.
42. Kwok, Pharmacogenomics 1, 95, (2000)

1. A method for diagnosing a genetic predisposition for a vascular disease comprising
providing a sample containing a nucleic acid derived from the gene GCH1; and
determining the presence or absence of a nucleotide polymorphism (SNP) of the gene GCH1 in the nucleic acid, wherein the SNP is selected from the group consisting of rs8007267 G>A, rs3783641 A>T, and rs10483639 C>G,
wherein the presence of at least one of said SNPs indicates a genetic predisposition for a vascular disease.

2. The method according to claim 1, wherein the nucleic acid is a genomic DNA, unprocessed RNA (hnRNA) or a cDNA derived therefrom.

3. The method according to claim 1, wherein the detection of the presence or absence of an SNP of the gene GCH1 in the nucleic acid is carried out by sequencing, hybridization, restriction-fragment-analysis, oligonucleotide ligation or allele-specific PCR.

4. The method according to claim 1, wherein the sample as provided is obtained from a body fluid.

5. The method according to claim 1, wherein the sample as provided is obtained from a tissue.

6. A kit for diagnosing a genetic predisposition for a vascular disease, comprising at least one probe and/or one primer for a detection of an SNP of the gene GCH1, wherein the SNP is selected from the group consisting of rs8007267 G>A, rs3783641 A>T, and rs10483639 C>G.

7. The kit according to claim 6, wherein the primer is a primer for an amplification reaction or a primer for a sequencing reaction.

8. The kit according to claim 7, wherein the primer for an amplification reaction is a primer according to SEQ ID NO:1 to 6, or the primer for a sequencing reaction according to SEQ ID NO: 7 to 9.

9. The method according to claim 1 which is used to diagnose a genetic predisposition for a vascular disease in a human.

10. (canceled)

11. The method, according to claim 1, used to diagnose a predisposition for coronary artery disease.


SEQUENCE LISTING (9 sequences)

SEQ ID NO: 1 - DNA, 18 bp, Artificial
  Primer
tggggtgagggttgagtt

SEQ ID NO: 2 - DNA, 21 bp, Artificial
  Primer
aatgttaacacaataggagcg

SEQ ID NO: 3 - DNA, 23 bp, Artificial
  Primer
gctatttgctttgtccacctcta

SEQ ID NO: 4 - DNA, 24 bp, Artificial
  Primer
aacctggaactgagaattgttcac

SEQ ID NO: 5 - DNA, 24 bp, Artificial
  Primer
atcctttcaatctggaactgactg

SEQ ID NO: 6 - DNA, 24 bp, Artificial
  Primer
gcattctaaaatcagggaaaatca

SEQ ID NO: 7 - DNA, 20 bp, Artificial
  Sequencing primer
cttgaatgactgaagtttgg

SEQ ID NO: 8 - DNA, 16 bp, Artificial
  Sequencing primer
cccacctgactcattt

SEQ ID NO: 9 - DNA, 20 bp, Artificial
  Sequencing primer
ggtgtgtgtatgtacaactt